(19) United States
(12) Patent Application Publication
(10) Pub. No.: US 2023/0010214 A1
(43) Pub. Date: Jan. 12, 2023

(54) DEODORIZING COMPOSITION

(71) Applicant: AQDOT LIMITED, Cambridge (GB)

(72) Inventors: Jose MARTINEZ-SANTIAGO, Cambridge (GB); Jessica LENDERYOU, Cambridge (GB); Roger COULSTON, Cambridge (GB)

(73) Assignee: AQDOT LIMITED, Cambridge (GB)

(21) Appl. No.: 17/784,839

(22) PCT Filed: Dec. 11, 2020
(86) PCT No.: PCT/GB2020/053175

§ 371 Date: Jun. 13, 2022

(30) Foreign Application Priority Data
Dec. 12, 2019 (GB) 1918266.6

(51) Int. Cl. A61K 8/49; A61K 8/26; A61K 8/27; A61Q 15/00

(52) U.S. Cl. CPC A61K 8/4946; A61K 8/26; A61K 8/27; A61Q 15/00

(57) ABSTRACT

A deodorizing composition includes cucurbituril and one or more first metal salts, the first metal salt including at least one metal cation with a valency of at least two and at least one anion, wherein the metal cation is selected from the group made of aluminium, zirconium, silver, titanium, iron, copper, zinc, and magnesium cations.

This invention provides a deodorizing composition comprising cucurbituril and one or more first metal salts, the first metal salt comprising at least one metal cation with a valency of at least two and at least one anion, wherein the metal cation is selected from the group consisting of aluminium, zirconium, silver, titanium, iron, copper, zinc, and magnesium cations.

The source of malodours often comprises complex mixtures of different compounds having diverse chemistries and odours. The malodour may be pungent and even minute quantities of such compounds can create uncomfortable impressions on consumers. Therefore, many strategies have been proposed to counteract malodours.

Preventive methods involve killing the bacteria that are often responsible for the generation of the malodourous compounds or inhibiting their growth, either by applying biocides, biostatic agents or controlling the microclimatic conditions in the locus where bacteria proliferate. These methods are used, for example, in the axilla or other parts of the human.

Eliminative methods involve using chemically reactive compounds that bind to the malodourous compounds and eliminate it by, for example, oxidation (burning) or ionization.

The disadvantage of all of the foregoing methods is that they involve agents that are not chemically or biologically inert, or environmentally neutral. In particular the use of biocides and reactive compounds is subject to health concerns. The by-products of combustion and ionization may also be toxic and hence undesirable.

Alternatively, fragrances can be used to mask or combine with the malodour in such a way that the perception of the malodour by the consumer is reduced. However, complex malodours are difficult to mitigate by using such sensory methods, because of the diversity of malodours involved.

Malodour suppression methods involve the use of absorbents and adsorbents. These materials are environmentally friendly and in most cases have no noticeable odour. Malodourous compounds are trapped in the pores of these materials and, therefore, their vapour pressure is depressed. However, these materials are generally not selective and may also bind desirable fragrance compounds. Furthermore, water vapour, such as those present in moist conditions, for example above 50% relative humidity, may displace the malodourous compounds which are then released back into the atmosphere.

Host compounds are a special class of absorbents, characterized in that each molecule has a well-defined cavity instead of a distribution of pores. Host-guest complexes have been used to counteract malodour for some time. Typical host compounds include cyclodextrin and cucurbituril.

U.S. Pat. No. 5,942,217 (The Procter & Gamble Company) discloses a composition comprising an aqueous solution of cyclodextrin, more generally referred to as a cyclic oligosaccharide, for use in absorbing malodour. Optionally the composition comprises a fragrance compound, but at a level that leaves a portion of the cyclodextrin molecules uncomplexed. However, the action of cyclodextrin on the perception of a malodour depends on the water activity in the system. This is a reflection of complex equilibria involving water and the malodourous compounds in the cyclodextrin cavity. Hence, the apparent host-guest binding constant may vary depending on the concentration of water in the system. This may lead to undesired release of malodour under moist conditions.

In U.S. Pat. No. 6,869,466 B2 (Unisearch Limited), a method is disclosed for binding a gas or a volatile compound to cucurbituril thereby to form an inclusion complex, as well as steps for releasing, at least a portion, of the bound gas or volatile compounds. Trapping an “unpleasant odour” is mentioned as an example of application. The formation of the host-guest complex is described as occurring using cucurbituril in both the dry state, for example with the cucurbituril host molecules supported on a solid surface, and when dissolved or dispersed in a liquid such as water.

WO 2014/077642 (Postech Academy-Industry Foundation) discloses a composition for removing odour, the composition comprising cucurbituril. The composition captures and removes odour, for example ammonium ion containing odour such as fish smell, by interaction between the hydrophobic cavity and hydrophilic inlets of cucurbituril and the malodourous compound. The composition optionally includes a fragrance compound. The composition is dissolved in an aqueous buffer of pH 7.2.

WO 2017/141029 (Aqdot Limited) discloses a composition comprising a mixture of cucurbiturils that is capable of binding various malodourous compounds, examples of which are selected amines, sulphur-containing compounds, and saturated and unsaturated alkyl and hydroxyalkyl carboxylic acids, under moist conditions, i.e., a relative humidity of higher than 40% at ambient temperature, for example at about 25 degrees centigrade.

Among these malodourous substances, short-chain saturated and unsaturated carboxylic acids and hydroxy acids emanating from the sweat, and, more particularly from the axilla sweat, are difficult to counteract.

There is therefore a need for providing improved odour control in areas where such short-chain carboxylic acids are produced, especially in the axilla region.

### SUMMARY OF THE INVENTION

In a first aspect of the invention a deodorizing composition is provided comprising, the deodorizing composition comprising cucurbituril and one or more first metal salts, the first metal salt comprising at least one metal cation with a valency of at least two and at least one anion, wherein the metal cation is selected from the group consisting of aluminium, zirconium, silver, titanium, iron, copper, zinc, and magnesium cations.

In a second aspect of the invention, use of the composition according to the first aspect of the invention for preventing or reducing malodour is provided, preferably the malodour is caused by a volatile organic compound, and preferably the volatile organic compound is an unsaturated organic acid, an unsaturated hydroxy acid or mixtures thereof.

In a third aspect of the invention, a method for preventing or reducing malodour is provided, the method comprising the step of applying the composition according to any one of claims 1 to 13 to a source of the malodour, preferably the malodour is caused by a volatile organic compound, preferably the volatile organic compound is an unsaturated organic acid, an unsaturated hydroxy acid or mixtures thereof, and preferably the source of the malodour is the axilla of a human.

The composition of the first aspect of the invention shows enhanced mitigation of the odour of a short chain acid found in sweat when compared to compositions comprising either cucurbiturils alone or first metal salts alone suggesting a surprising synergy between cucurbituril and first metal salt.

Consequently, the composition of the first aspect of the invention is useful as a malodour counteracting composition. Where the first metal salt is also an antiperspirant active, the composition may then also have antiperspirant activity.

### DETAILED DESCRIPTION OF THE INVENTION

In a first aspect of the invention a deodorizing composition is provided comprising, the deodorizing composition comprising cucurbituril and one or more first metal salts, the first metal salt comprising at least one metal cation with a valency of at least two and at least one anion, wherein the metal cation is selected from the group consisting of aluminium, zirconium, silver, titanium, iron, copper, zinc, and magnesium cations.

Preferably the cucurbituril is selected from the group consisting of curcubit[5]uril, curcubit[6]uril, curcubit[7]uril, curcubit[8]uril, curcubit[9]uril, curcubit[10]uril, curcubit[11]uril, and mixtures thereof, more preferably the cucurbituril is selected from the group consisting of curcubit[6]uril, curcubit[7]uril, curcubit[8]uril, and mixtures thereof.

Preferably the at least one anion is selected from the group consisting of hydroxide, chloride, bromide, acetate, lactate, glycinate, ricinoleate, nitrate, sulphate, oxalate, citrate, phosphate and borate anions.

In one embodiment, the first metal salt is a polynucleated metal salt. Typical polynucleated metal salts are poly-aluminium salts often used as coagulants. Polynucleated metal salts comprise one or more polyvalent cation. They may, additionally, comprise one or more monovalent metal cation, such as lithium, sodium or potassium cations. Polynucleated metal salts optionally comprise more than one anion. In addition, polynucleated metal salts optionally comprise water of hydration and/or amino acids, such as glycine, alanine, valine, serine, leucine, or aminobutyric acid.

In one embodiment, the polynucleated metal salt is a poly-aluminium salt, more particularly aluminium chlorohydrate (ACH), with chemical formula Al₂(OH)₅Cl, or poly-aluminium chlorhydrate, with chemical formula [Al₂(OH)₅Cl)]ₓ, and/or poly-aluminium chloride (PACL), with chemical formula [Al₂(OH)₃Cl₃)]ₓ, wherein x is at least 1.

In another embodiment, the first metal salt is aluminium chlorohydrex PG (CAS number 245090-52-2) or chlorohydrex PEG (CAS number 242812-76-6), wherein the water molecules coordinated to the aluminium chlorohydrate have been displaced by either propylene glycol or polyethylene glycol, resulting in a less polar complex.

In another embodiment, the metal salt is alum with chemical formula Al₂ (SO₄)₃.18H₂O.

In yet another embodiment, the polynucleated salt is a mixed salt, such as aluminium zirconium mixed salt, aluminium zinc mixed salts, aluminium magnesium mixed salts, potassium alum (KAl(SO₄)₂, although commonly encountered as the dodecahydrate KAl(SO₄)₂.12H₂O, ammonium alum, sodium alum, and the like.

In a particular embodiment, the mixed salt comprises aluminium and zirconium. Typical aluminium zirconium salts include aluminium zirconium tetrachlorohydrate or aluminium zirconium tetrachlorohydrex glycine (aluminium zirconium tetrachlorohydrex gly, CAS number 134910-86-4).

Thus preferably, the first metal salt is selected from the group consisting of aluminium chlorohydrate, aluminium chloride, aluminium sulfate, aluminium sesquichlorohydrate, sodium aluminium chlorohydroxy lactate, aluminium zirconium tetrachlorohydrate, aluminum zirconium tetrachlorohydrex gly, aluminium chlorohydrex PG, aluminium chlorohydrex PEG, zinc ricinoleate, zinc glycinate, zinc oxide, zinc salicylate, zinc resorcinoleate, zinc-containing polymers, a complex of zinc and polyitaconic acid (available, for example, as Itaconix® ZINADOR™ 22L), cupric sulfate and silver sulfate.

The composition of the first aspect of the invention may be in the form of a solid, a gel, a paste or a liquid. Preferably the composition has a pH of 2 to 9, more preferably 3 to 8, however if the composition is anhydrous, the pH is preferably 2 to 9, more preferably 3 to 8 when in contact with an external source of water, i.e., not from the composition, from, for example, the locus of application, for example from sweat. Thus the composition may be anhydrous or hydrous.

Preferably the pH of the composition is kept within the desired pH range by mean of a buffer, such as a phosphate buffer or a citrate buffer.

In one embodiment, the composition is in the form of a liquid, the liquid comprising a polar liquid, preferably wherein the polar liquid is selected from the group consisting of water, glycols such as propylene glycol, polyols such as glycerol, and mixtures thereof.

In another embodiment, the composition comprises water and the cucurbituril and first metal salt are dispersed to form a transparent, hazy or turbid dispersion in the form of a liquid or gel.

The cucurbituril particle size distribution in a composition of the first aspect of the invention wherein the cucurbituril is dispersed rather than solubilised in the remainder of the composition is typically characterized by a D(10) of from 4 to 7 micrometers, a D(50) of from 8 to 24 micrometers and a D(90) of from 25 to 50 micrometers, wherein D(10), D(50) and D(90) refers to the percentage of particles under the reported particle size (diameter). D(50) is usually taken as the volume-average size of a monomodal particle size distribution as measured by static light scattering.

The composition preferably comprises 0.1-5, more preferably 0.25-3, and most preferably 0.5-1.25% w/w cucurbituril.

The composition preferably comprises 1-50, more preferably 2.5-45% w/w one or more first metal salts.

Preferably the weight ratio between cucurbituril and the one or more first metal salts is 0.002-0.2, more preferably 0.01-0.1.

Compositions according to the first aspect of the invention in the form of a liquid may separate into an essentially transparent supernatant comprising soluble cucurbituril, and a solid, usually powder-like precipitate, comprising insoluble cucurbituril. Both supernatant and precipitate may be used as such or together to counteract a malodour.

The term “malodour” refers to unpleasant odours which are frequently encountered in everyday life and have a variety of origins. Typical malodours include odours that emanate from uncontrolled industrial activity, from human and pet body such as perspiration and excretion, from kitchen and food processing, from tobacco smoke, and from mould. Some of the most disturbing malodours for the human originate from sweat, faeces, urine, a wet pet, cooking especially from garlic, cabbage, fish and onion. Malodourous compounds may be fatty acid and fatty acid derivatives present in consumer products, for example in soaps, detergents, shampoos, and conditioners. Other examples of particularly undesirable malodours are those produced by depilatory creams (sulphur compounds).

In order to maintain insoluble cucurbituril in a dispersed, self-suspending form which is more easily applied on substrates, preferably the composition of the first aspect of the invention additionally comprises one or more dispersing agent.

The dispersing agent may be selected from the group consisting of water-soluble polymers, such as polysaccharides; polymer surfactants, such as amphiphilic linear or branched block copolymers; and surfactants, such as anionic, cationic, amphoteric, zwitterionic and non-ionic surfactants, preferably the dispersing agent is a non-ionic surfactant or a zwitterionic surfactant.

The dispersing agent may be selected from the group consisting of a zwitterionic surfactant, a second metal salt, an ammonium salt, and a polyhydroxylated organic compound, preferably the zwitterionic surfactant is an alkylamido-betaine and/or an alkyldimethyl-betaine, preferably the zwitterionic surfactant is cocamidopropylbetaine (CAS number 61789-40-0) or cocodimethyl betaine (CAS number 68424-94-2), wherein the second metal salt comprises only one or more metal cation with a valency of one, preferably the second metal salt is sodium chloride, potassium chloride, or caesium chloride, preferably the ammonium salt is ammonium chloride, preferably the polyhydroxylated organic compound is a sugar, and preferably the sugar is glucose or sucrose.

Preferably the composition of the first aspect of the invention comprises a zwitterionic surfactant in combination with at least one of a second metal salt, ammonium salt, and a polyhydroxylated organic compound, most preferably the composition comprises a zwitterionic surfactant in combination with a second metal salt.

Preferably the composition of the first aspect of the invention comprises 0.1 to 2, preferably 0.5 to 1% w/w dispersing agent.

Preferably the composition of the first aspect of the invention additionally comprises at least one fragrance compound, preferably wherein at least one fragrance compound is complexed with cucurbituril.

Extensive lists of suitable fragrance compounds may be found under www.thegoodscentscompany.com or in text books such as Stefen Arctander, Perfume and Flavour Chemicals (Aroma Chemicals), Vol. I and II (Allured Publishing 1982) and Stefen Arctander, Perfume and Flavour Materials of Natural Origin (Allured Publishing 1994, 2003).

The composition of the first aspect of the invention may also include one or more additives known to those skilled in the art. Thus the composition may also comprise additives, such as gelling agents, viscosity modifiers, dyes, pigments, sequestrants, antioxidants and preservatives.

In one embodiment, the composition further comprises one or more compounds (including polymers) useful in counteracting malodour, in addition to cucurbituril and the first metal salt. Suitable compounds are known to the skilled person and include other host molecules. For example, other members of the cavitand family, which includes cyclodextrin, calixarene and crown ether, could be used in combination with cucurbituril and the first metal salt. A further suitable compound includes charcoal.

The composition of the first aspect of the invention may be a consumer product, preferably the consumer product is a personal care product, and preferably the personal care product is a deodorant product or an antiperspirant deodorant product.

The composition of the first aspect of the invention can be provided in a multitude of forms and formats. In one particular embodiment, the composition may be provided in powder or granulate form, tablets or single-dose units, in emulsion or micro-emulsion form, in solution or as a dispersion in a liquid, in a super-critical liquid or as a compressed gas, adsorbed on a substrate, for example on a fabric, a non-woven pad, an adsorbent, and the like, in the form of a gel or a solid, for example, a stick form, or in spray form.

The composition of the first aspect of the invention may be applied as a detergent, a cleansing composition, a shampoo, a softener, a softener sheet, a conditioner, a refresher, an air freshener, a deodorizing composition, a personal deodorant, an antiperspirant, a cosmetic product, a fine fragrance, a body mist, a candle, a hard surface cleaner, a cleansing wipe or mop, a soap, a styling gel, a humidity absorber, an air filtration device, a finishing product, a diaper or sanitary product, and the like. Methods of applying the composition in these different forms are well known to the person skilled in the art.

The composition may also be used to provide malodour counteracting properties to textiles, to functional textiles and to textile finishing product; to air and various materials, such as paper, wood, plastics, stone, ceramics, metals, metal wool, wool, fibres, foams, filter material, absorbents, adsorbents, plasters, paints, inks, and the like.

The composition may also be admixed with or incorporated into perfume oil, before addition to a product. If the composition is added to perfume oil, the resulting mixture may then be microencapsulated by any methods known in the art, such as by spray drying, spray granulation, matrix particle formation, core-shell encapsulation, and the like.

One embodiment of the composition of the first aspect of the invention may be prepared in the following way:

a) Optionally admixing a dispersing agent with water;

b) Admixing one or more first metal salt with water or with the mixture obtained in step a);

c) Dispersing cucurbituril into the mixture obtained in step b);

and optionally adding sodium chloride or potassium chloride into any of the mixture obtained in step a), b), or c).

The skilled person would be able to prepare other embodiments of the composition of the first aspect of the invention.

In a second aspect of the invention, use of the composition according to the first aspect of the invention for preventing or reducing malodour is provided, preferably the malodour is caused by a volatile organic compound, and preferably the volatile organic compound is selected from the group consisting of a saturated organic acid, an unsaturated organic acid, a saturated hydroxy acid, an unsaturated hydroxy acid, or mixtures thereof.

The saturated and unsaturated organic acids and hydroxy acids may be short (C₂-C₅) and medium (C₆-C₁₁) chain acids, such as propionic acid, butyric acid, isobutyric acid, valeric acid, isovaleric acid, hexanoic acid, caprylic acid, capric acid, 3-hydroxy-methylhexanoic acid), and (E)-3-methyl-2-hexenoic acid.

In a third aspect of the invention, a method for preventing or reducing malodour is provided, the method comprising the step of applying the composition according to the first aspect of the invention to a source of the malodour, preferably the malodour is caused by a volatile organic compound, preferably the volatile organic compound is selected from the group consisting of a saturated organic acid, an unsaturated organic acid, a saturated hydroxy acid, an unsaturated hydroxy acid, or mixtures thereof, and preferably the source of the malodour is the axilla of a human.

In one embodiment, the malodour to be counteracted is a complex malodour caused by more than one malodourous compound which may or not be a volatile organic compound.

In another embodiment, the complex malodour comprises malodourous compounds having a diversity of chemical functional groups.

Cucirbit[5]uril, having a cavity volume of 82 Å³, preferably binds small gaseous malodourous compounds, such as acetylene, hydrogen sulphide and carbon disulphide.

Larger malodourous compounds comprising O-, N- and S-heteroatoms preferably bind to larger cucurbiturils, for example cucirbit[7]uril and cucirbit[8]uril).

The malodourous compounds may be selected from, but not limited to: Nitrogen- and sulphur-containing molecules, such as allyl amine; methyl amine; ethyl amine; cyclobutyl amine (cyclobutanamine, urine), cyclopentyl amine (cyclopentanamine); cyclohexyl amine (cyclohexanamine); cycloheptyl amine (cyclobutanamine); isopropylamine; butylamine; dibutylamine (N-butyl-1-butanamin); dimethyl ethanolamine (2-(dimethylamino)ethanol); methyl ethanolamine (2-(methylamino)ethanol); diethyl ethanolamine (2-(diethylamino)ethanol); diethylamine (N-methylethanamine (fishy smell)); dipropyl amine (N-propyl-1-propanamine); diisopropylamine (N-isopropyl-2-propanamine); dimethyl acetamide (N,N-dimethylacetamide); ethyl methylamine (N-methylethanamine); ethyl propylamine (N-ethylpropanamide); trimethyl amine (fishy smell); triethylamine (fishy smell); ethylene diamine (1,2-ethanediamine (musty ammoniacal smell)); propylene diamine (1,3-propanediamine); tetramethylenediamine (1,4-butanediamine, putrescine (foul smell)); ethylene imine (aziridine (ammoniacal smell)); morpholine (fishy smell); ethyl morpholine (4-ethylmorpholine (sour smell)); pyrrolidine (semen smell); methyl ethyl pyridine (2-ethyl-3-methylpyridine); pyridine (burnt, sickening smell); vinyl pyridine (4-vinylpyridie (nauseating smell)); skatole (3-methylindole (faecal smell)); indole (faecal smell); cadaverine (pentane-1,5-diamine (putrid smell)); hydrogen sulphide (rotten egg smell); allyl disulphide (3-(allyldisulfanyl)-1-propene (garlic smell)); ethyl isothiocyanate (isothiocyanatoethane (pungent, mustard, garlic smell)); ally isothiocyanate (3-isothiocyanatoprop-1-ene (sulphurous smell)); allyl mercaptan (2-propene-1-thiol (garlic, sulphurous smell)); allyl sulphide (3-(allylsulfanyl)-1-propene (sulphurous smell)); diallyl sulphide (3-(allylsulfanyl)-1-propene (sulphurous smell)); dimethyl disulphide ((methylsulfanyl)ethane (unpleasant, garlic smell)); dimethyl trisulphide (dimethyltrisulfane (foul smell)); diethyl sulphide ((ethylsulfanyl)ethane (sulphurous)); butyl sulphide (1-(butylsulfanyl)butane (garlic, violet smell)); diethyl trisulfide (diethyltrisulfane (foul, garlic smell)); ethyl methyl disulphide ((methylsulfanyl)ethane (sulphurous smell)); phenyl sulphide (1,1′-sulfanediyldibenzene (sulphurous smell)); ethyl mercaptan (1.ethanethiol (sulphurous smell)); amyl mercaptan (1-pentanethiol); isoamyl mercaptan (3-methylbutane-1-thiol (sulphurous, onion smell)); butyl mercaptan (1-butanethiol (skunk-like smell)); isobutyl mercaptan (2-methylpropane-1-thiol (sulphurous, mustard smell)); dodecyl mercaptan (1-dodecanethiol); carbon disulphide (methanedithione (disagreeable, sweet smell)); dimethyl trithiocarbonate (dimethyl carbonotrithioate); and thiophenol mercaptan;

Oxygen-containing five-member ring molecules, such as sotolone; and nor-sotolone;

Saturated and unsaturated alkyl and hydroxyalkyl carboxylic acids, such as acetic acid, propionic acid, butyric acid, iso-valeric acid, n-valeric acid, 2-methyl-butyric acid, 3-methyl-2-hexenoic acid, and 3-methyl-3-hydroxy hexanoic acid.

### EXAMPLES

### Example 1 Malodour-Counteracting Composition Comprising a Mixture of Cucurbiturils and Aluminium Chlorohydrate

A 0.5% w/w aqueous solution of isovaleric acid, a malodorous compound produced from sweat by skin bacteria, was added to an aqueous solution of 1% w/w mixture of cucurbiturils (hereinafter referred to CB[n]) and 10% w/w aluminium chlorohydrate (Making Cosmetics). The mixture of cucurbiturils consisted of 58% w/w cucurbit[6]uril, 27% w/w cucurbit[7]uril and 15% w/w cucurbit[8]uril.

The % reduction of volatile isovaleric acid compared to a water control was measured using headspace-gas chromatography. In particular an Agilent gas chromatograph (7890B) with headspace sampler (7697A) and flame ionisation detector was used together with an Agilent HP-5 column ((5%-phenyl)-methylpolysiloxane; product code: 19091J-416). The headspace temperature was set to 90° C. and the inlet to 250° C. The oven conditions were: 50° C. for 1 min; 10° C./min to 65° C. and hold for 4 min; 100° C./min to 250° C. and hold for 1 minute. Once prepared, the vials were then placed in the headspace autosampler. Triplicate analyses of these samples were run.

The samples were:
(a) The CB[n] and isovaleric acid control: formulated by adding 0.4 mL of isovaleric acid solution to a 20 mL glass headspace vial that contained 2 g of a 1% w/w CB[n] aqueous solution.
(b) The aluminium chlorohydrate and isovaleric acid control: formulated by adding 0.4 mL of isovaleric acid solution to a 20 mL glass headspace vial containing 2 g of a 10% w/w aluminium chlorohydrate aqueous solution.
(c) The CB[n] and aluminium chlorohydrate and malodour test: formulated by adding 0.4 mL of isovaleric acid solution to a 20 mL glass headspace vial that contained 2 g of a 1% w/w CB[n] and 10% w/w aluminium chlorohydrate aqueous solution.

The results are summarised in Table 1 and show that rather surprisingly in the presence of aluminium chlorohydrate, isovaleric acid, a model malodorous compound, is counteracted by CB[n] to a higher degree compared to the composition without aluminium chlorohydrate. This is a result contrary to what the skilled person would have expected which is the formation of a host-guest interaction between the aluminium cations with cucurbituril molecules which would result in a reduction in the ability of CB[n] to reduce isovaleric acid in the headspace above the solution.

TABLE 1
Percentage reduction of isovaleric acid using head space analysis
in the presence of an aqueous solution of 1% w/w CB[n]
and 10% w/w aluminium chlorohydrate compared to a water control.

| | % Reduction |
|---|---|
| | (± % std dev) |
| CB[n] | 25% (±4%) |
| Aluminium chlorohydrate | 33% (±2%) |
| CB[n]/aluminium chlorohydrate | 52% (±2%) |

### Example 2 Malodour-Counteracting Composition Comprising a Mixture of Cucurbiturils and Metal Salts

Example 1 was repeated using each of aluminium sesquichlorohydrate (Reach 301, ex Elementis), aluminium zirconium tetrachlorohydrate-glycine (Reach AZP-908, ex Elementis), and zinc oxide in place of aluminium chlorohydrate.

The results are summarised in Table 2 and show that rather surprisingly, in the presence of all the metal salts, isovaleric acid is counteracted by CB[n] to a higher degree compared to the compositions without metal salts.

TABLE 2
Percentage reduction of isovaleric acid (± % std dev) using
head space analysis in the presence of an aqueous solution of
1% w/w CB[n] and 10% w/w metal salts compared to a water control.

| % reduction | Without | With |
|---|---|---|
| (± % std dev) | CB[n] | CB[n] |
| Aluminium chlorohydrate | 33% (±2%) | 52% (±2%) |
| Aluminium sesquichlorohydrate | 24% (±2%) | 48% (±2%) |
| Aluminium zirconium | 66% (±2%) | 75% (±2%) |
| tetrachlorohydrate-glycine | | |
| Zinc oxide | 80% (±0%) | 96% (±1%) |

### Example 3 Malodour-Counteracting Composition Comprising a Mixture of Cucurbiturils, Aluminium Chlorohydrate and Dispersing Agents

Example 1 was repeated but including 0.8% w/w cocamidopropyl betaine (CAPB) (ex Mistral) and 1% w/w NaCl as dispersing agents in the malodour-counteracting composition.

The results are summarised in Table 3 and show that the performance of the composition is unaffected by the presence of CAPB and NaCl as dispersing agents. The dispersing agents had a significant effect on reducing sedimentation of cucurbituril.

TABLE 3
Percentage reduction of isovaleric acid using head space analysis
in the presence of an aqueous solution of 1% w/w CB[n],
10% w/w aluminium chlorohydrate, 0.8% w/w cocamidopropyl betaine
(CAPB) and 1% w/w NaCl compared to a water control.

| | % Reduction |
|---|---|
| | (± % std dev) |
| CB[n] | 25% (±4%) |
| Aluminium chlorohydrate | 33% (±2%) |
| CB[n]/aluminium chlorohydrate | 52% (±2%) |
| CB[n]/aluminium chlorohydrate/CAPB | 51% (±1%) |
| CB[n]/aluminium chlorohydrate/CAPB/NaCl | 47% (±1%) |

### Example 4 Commercial Deodorant Antiperspirant Formulations

Example 1 was repeated using commercially available deodorant antiperspirant formulations with 1% w/w CB[n] added instead of the malodour-counteracting composition used in Example 1.

The results are summarised in Table 4 and show that surprisingly isovaleric acid is further counteracted by CB[n] when added into commercial deodorant antiperspirant formulations all of which comprised aluminium chlorohydrate.

TABLE 4
Percentage reduction of isovaleric acid (± % std dev)
using head space analysis in the presence of commercial
deodorant antiperspirant formulations comprising 1% w/w CB[n].

| | Without | With |
|---|---|---|
| | CB[n] | CB[n] |
| Dove Original Antiperspirant Roll | 39% (±4%) | 46% (±3%) |
| On Deodorant | | |
| L′Oreal Men Expert Pro Fresh | 41% (±3%) | 54% (±4%) |
| Extreme Roll On Deodorant | | |
| Lynx Antiperspirant Deodorant | 43% (±2%) | 55% (±4%) |
| Roll On Africa | | |
| Nivea Protect & Care Roll On 0% alcohol | 47% (±2%) | 56% (±3%) |
| Sanex Dermo Sensitive Roll On Deodorant | 47% (±2%) | 55% (±2%) |

### Example 5 Malodour-Counteracting Composition Comprising a Mixture of Cucurbiturils and Aluminium Chlorohydrate at Different Concentrations

Example was repeated testing malodour-counteracting compositions comprising 1% w/w CB[n] and from 0 to 30% w/w aluminium chlorohydrate and malodour-counteracting compositions comprising 10% w/w aluminium chlorohydrate and 0 to 2% w/w CB[n].

The results are summarised in Table 5 and Table 6 for a fixed amount of CB[n] (1% w/w) and a fixed amount of aluminium chlorohydrate (10% w/w) respectively. Table 5 shows that at all levels aluminium chlorohydrate increases the percentage reduction isovaleric acid in the head space seen with CB[n] alone and furthermore the reduction increases with increases in the amount of aluminium chlorohydrate. Table 6 shows a similar result with increasing amounts of CB[n].

TABLE 5
Percentage reduction of isovaleric acid (± % std dev)
using head space analysis in the presence of an aqueous
solution of 1% w/w CB[n] and 0-30% w/w aluminium chlorohydrate.

% Reduction (± % std dev)

| Aluminium | Without | With CB[n] |
|---|---|---|
| chlorohydrate/% | CB[n] | (1% w/w) |
| 0 | 0 | 26% (±3%) |
| 5 | 28% (±1%) | 43% (±3%) |
| 10 | 36% (±3%) | 52% (±1%) |
| 15 | 41% (±3%) | 54% (±2%) |
| 20 | 48% (±2%) | 61% (±2%) |
| 30 | 55% (±2%) | 67% (±2%) |

TABLE 6
Percentage reduction of isovaleric acid (± % std dev)
using head space analysis in the presence of an aqueous solution
of 10% w/w aluminium chlorohydrate and 0-2.00% w/w CB[n].

% reduction (± % std dev)

| | Without aluminium | With aluminium |
|---|---|---|
| CB [n]/% | chlorohydrate | chlorohydrate (10% w/w) |
| 0.00 | 0 | 34% (±2%) |
| 0.50 | 11% (±2%) | 44% (±3%) |
| 0.75 | 17% (±3%) | 48% (±2%) |
| 1.00 | 24% (±3%) | 53% (±1%) |
| 1.50 | 37% (±4%) | 60% (±2%) |
| 2.00 | 53% (±3%) | 67% (±1%) |

1. A deodorizing composition comprising cucurbituril and one or more first metal salts, the first metal salt comprising at least one metal cation with a valency of at least two and at least one anion, wherein the metal cation is selected from the group consisting of aluminium, zirconium, silver, titanium, iron, copper, zinc, and magnesium cations.

2. The composition according to claim 1, wherein the cucurbituril is selected from the group consisting of curcubit[5]uril, curcubit[6]uril, curcubit[7]uril, curcubit[8]uril, curcubit[9]uril, curcubit[10]uril, curcubit[11]uril, and mixtures thereof.

3. The composition according to claim 1, wherein the at least one anion is selected from the group consisting of hydroxide, chloride, bromide, acetate, lactate, glycinate, ricinoleate, nitrate, sulphate, oxalate, citrate, phosphate and borate anions.

4. The composition according to claim 1, wherein the first metal salt is selected from the group consisting of aluminium chlorohydrate, aluminium chloride, aluminium sulfate, aluminium sesquichlorohydrate, sodium aluminium chlorhydroxy lactate, aluminium zirconium tetrachlorohydrate, aluminum zirconium tetrachlorohydrex gly, aluminium chlorohydrex PG, aluminium chlorohydrex PEG, zinc ricinoleate, zinc glycinate, zinc oxide, zinc salicylate, zinc ricinoleate, zinc salt of ricinoleic acid, sodium salt of poly(itaconic) acid, potassium salt of poly(itaconic) acid, zinc salt of poly(itaconic) acid, zinc-containing polymers, a complex of zinc and polyitaconic acid, cupric sulfate and silver sulfate.

5. The composition according to claim 1, wherein the composition is has a pH of 2 to 9, or if anhydrous has a pH of 2 to 9, when in contact with an external source of water.

6. The composition according to claim 1, wherein the composition comprises 0.1-5% w/w cucurbituril.

7. The composition according to claim 1, wherein the composition comprises 1-50% w/w one or more first metal salts.

8. The composition according to claim 1, wherein the weight ratio between cucurbituril and the one or more first metal salts is 0.002-0.2.

9. The composition according to claim 1, additionally comprising one or more dispersing agent.

10. The composition according to claim 9, wherein the dispersing agent is selected from the group consisting of a zwitterionic surfactant, a second metal salt, an ammonium salt, and a polyhydroxylated organic compound, wherein the second metal salt comprises only one or more metal cation with a valency of one.

11. The composition according to claim 10, wherein the composition comprises a zwitterionic surfactant in combination with at least one of a second metal salt, ammonium salt, and a polyhydroxylated organic compound.

12. The composition according to claim 1, additionally comprising at least one fragrance compound.

13. The composition according to claim 1, which is a personal care consumer product.

14. A method comprising applying the composition according to claim 1 for preventing or reducing malodour.

15. A method for preventing or reducing malodour comprising the step of applying the composition according to claim 1 to a source of the malodour.
